(19) United States
(12) Patent Application Publication
(10) Pub. No.: US 2017/0015607 A1
(43) Pub. Date: Jan. 19, 2017

(54) PROCESSES FOR THE PRODUCTION OF Z 1,1,1,4,4,4 HEXAFLUORO 2-BUTENE

(71) Applicant: THE CHEMOURS COMPANY FC, LLC, Wilmington, DE (US)

(72) Inventors: IVAN SERGEYEVICH BALDYCHEV, WILMINGTON, DE (US); STEPHAN M. BRANDSTADTER, PHILADELPHIA, PA (US); MARIO JOSEPH NAPPA, NEWARK, DE (US); SHENG PENG, HOCKESSIN, DE (US)

(21) Appl. No.: 15/124,738

(22) PCT Filed: Mar. 18, 2015
(86) PCT No.: PCT/US15/21147

§ 371 Date: Sep. 9, 2016

Related U.S. Application Data
(60) Provisional application No. 61/968,467, filed on Mar. 21, 2014.
(60) Provisional application No. 62/018,048, filed on Jun. 27, 2014.

(51) Int. Cl. C07C 17/04

(52) U.S. Cl. CPC C07C 17/04

(57) ABSTRACT

Processes are provided for (i) producing Z-1,1,1,4,4,4-hexafluoro-2-butene (Z—CF₃CH═CHCF₃) from E-1,1,1,4,4,4-hexafluoro-2-butene (E-CF₃CH═CHCF₃), comprising the steps of (a) reacting the E-1,1,1,4,4,4-hexafluoro-2-butene (E-CF₃CH═CHCF₃) with chlorine to form dichloro-1,1,1,4,4,4-hexafluorobutane (CF₃CHClCHClCF₃), (b) recovering the dichloro-1,1,1,4,4,4-hexafluorobutane from step (a), (c) reacting the recovered dichloro-1,1,1,4,4,4-hexafluorobutane with base to form 1,1,1,4,4,4-hexafluoro-2-butyne (CF₃≡CCF₃), (d) recovering the 1,1,1,4,4,4-hexafluoro-2-butyne from step (c) and (e) reacting the recovered hexafluoro-2-butyne with hydrogen to form said Z-1,1,1,4,4,4-hexafluoro-2-butene, optionally wherein said E-1,1,1,4,4,4-hexafluoro-2-butene starting material is obtained by converting 1,1,1,4,4,4-hexafluoro-2-butane (F₃CHCl₂) to a mixture of Z-1,1,1,4,4,4-hexafluoro-2-butene and E-1,1,1,4,4,4-hexafluoro-2-butene and recovering the E-1,1,1,4,4,4-hexafluoro-2-butene from said mixture, whereby the E-1,1,1,4,4,4-hexafluoro-2-butene used in step (a) is the recovered E-1,1,1,4,4,4-hexafluoro-2-butene, and (ii) step (a) as a subcombination of the process (i).

### FIELD OF THE INVENTION

This invention relates to the production of Z-1,1,1,4,4,4-hexafluoro-2-butene, which in one embodiment uses E-1,1,1,4,4,4-hexafluoro-2-butene as the starting material.

### BACKGROUND OF THE INVENTION

U.S. Pat. No. 8,436,216 discloses the preparation of haloolefins that have low ozone depletion and low global warming attributes desired for such application as refrigerants and foam expansion agents. In '216, 2,2-dichloro-1,1,1-trifluoroethane (HCFC-123) is catalytically converted to a mixture of Z-1,1,1,4,4,4-hexafluoro-2-butene and E-1,1,1,4,4,4-hexafluoro-2-butene, Z-1336mzz and E-1336mzz, respectively. The mixture is about 50:50 of each isomer, wherein the Z-isomer has the cis configuration, and the E-isomer has the trans configuration. These isomers are separated from one another by distillation. The E-isomer boils at about 7° C. and the Z-isomer boils at about 33° C. at ambient temperature (15-25° C.) and pressure (0.7 to 1 Bar).

Because the Z-isomer is liquid at ambient temperature and pressure, the Z-isomer is generally preferred over the E-isomer.

The problem is how to obtain greater value from the E-isomer.

### SUMMARY OF THE INVENTION

The present invention solves this problem by in one embodiment providing an integrated process for obtaining Z-1,1,1,4,4,4-hexafluoro-2-butene from E-1,1,1,4,4,4-hexafluoro-2-butene, i.e. the Z-isomer from the E-isomer.

The integrated process for producing Z-1,1,1,4,4,4-hexafluoro-2-butene (Z—CF₃CH═CHCF₃) comprises the steps of
(a) reacting E-1,1,1,4,4,4-hexafluoro-2-butene (E-CF₃CH═CHCF₃) (E-1336mzz) with chlorine to form dichloro-1,1,1,4,4,4-hexafluorobutane (CF₃CHClCHClCF₃)(HCFC-336mdd),
(b) recovering the dichloro-1,1,1,4,4,4-hexafluorobutane from step (a),
(c) reacting the recovered dichloro-1,1,1,4,4,4-hexafluorobutane with base to form 1,1,1,4,4,4-hexafluoro-2-butyne (CF₃C≡CCF₃),
(d) recovering the hexafluoro-2-butyne from step (c),
(e) reacting the recovered 1,1,1,4,4,4-hexafluoro-2-butyne with hydrogen to form said Z-1,1,1,4,4,4-hexafluoro-2-butene.

This process can be supplemented by the step of recovering the Z-1,1,1,4,4,4-hexafluoro-2-butene (Z-1336mzz) from step (e).

In one aspect of integrated process of the present invention, the E-1,1,1,4,4,4-hexafluoro-2-butene starting material is obtained from any source.

In another aspect of the integrated process of the present invention, the E-1,1,1,4,4,4-hexafluoro-2-butene starting material is obtained from the mixture of this E-isomer (E-1336mzz) with Z-1,1,1,4,4,4-hexafluoro-2-butene, the Z-isomer (Z-1336mzz), such as is obtained by the process of U.S. Pat. No. 8,436,216, referred to above. According to this aspect, the integrated process is back-integrated by the E-1,1,1,4,4,4-hexafluoro-2-butene being obtained by converting 2,2-dichloro-1,1,1-trifluoroethane (HCFC-123) to a mixture of Z-1,1,1,4,4,4-hexafluoro-2-butene and E-1,1,1,4,4,4-hexafluoro-2-butene (E/Z-1336mzz) and recovering the E-1,1,1,4,4,4-hexafluoro-2-butene from said mixture, whereby the E-1,1,1,4,4,4-hexafluoro-2-butene used in step (a) of the integrated process is this recovered E-1,1,1,4,4,4-hexafluoro-2-butene.

Another embodiment of the present invention is the process for obtaining dichloro-1,1,1,4,4,4-hexafluorobutane, comprising reacting E-1,1,1,4,4,4-hexafluoro-2-butene with chlorine. This is a subcombination, namely step (a), of the integrated process of the present invention. This subcombination can also include the recovery step (b).

In still another aspect of the integrated process of the present invention, the E-1,1,1,4,4,4-hexafluoro-2-butene starting material used in step (a) is obtained by (i) reacting 3,3,3-trifluoroprop-1-ene with carbon tetrachloride to form 2,4,4,4-tetrachloro-1,1,1-trifluorobutane and (ii) fluorinating said 2,4,4,4-tetrachloro-1,1,1-trifluorobutane to form said E-1,1,1,4,4,4-hexafluoro-2-butene. This is another back integration of the process comprising steps (a)-(e) described above.

The foregoing general description and the following detailed description are exemplary and explanatory only and are not restrictive of the invention as defined in the appended claims.

### DESCRIPTION OF THE PREFERRED EMBODIMENTS

The process of the present invention comprising steps (a) to (e), and optionally the conversion of HCFC-123 to the mixture E/Z-1336mzz, followed by recovery of the E-1336mzz to serve as the starting material for step (a) reaction, is an integrated process in that the desired reaction product of one reaction step after recovery serves as the starting material (reactant) in the next reaction step of the sequence of reactions constituting the integrated process. The same is true when the reactions (i) and (ii) are conducted to provide the E-1336mzz starting material for step (a).

The recovering steps between reaction steps (b) and (d) and the recovering of E-1,1,1,4,4,4-hexafluoro-2-butene from the mixture of Z-1,1,1,4,4,4-hexafluoro-2-butene and E-1,1,1,4,4,4-hexafluoro-2-butene and the Z-1,1,1,4,4,4-hexafluoro-2-butene from the reaction of step (e), are conducted to sufficiently isolate the desired reaction product to make it available for its intended use, either as a starting material for the next reaction step or in the case of recovery of Z-1,1,1,4,4,4-hexafluoro-2-butene, useful as a refrigerant or foam expansion agent. The details of the recovery step will depend on the compatibility of the reaction system producing the desired reaction product with the reaction system of the next reaction step. For example, if the reaction product is produced in a reaction medium that is different from or incompatible with the succeeding reaction step, then the recovery step will include separation of the desired reaction product from its reaction medium. This separation may occur simultaneously with the reacting step when the desired reaction product is volatile under the reaction conditions. The volatilization of the desired reaction product can constitute the isolation and thereby the recovery of the desired reaction product. If the vapors include other materials intended for separation from the desired reaction product, the desired reaction product can be isolated by selective distillation.

The recovery steps preferably separate the desired reaction product from any reaction promoter used to make the desired reaction product.

Each of the reaction steps described above is preferably carried out in the presence of a reaction promoter that is effective to produce the desired reaction product in useful selectivity. Examples of reaction promoters include catalysts and photoinitiators, i.e. initiation of the reaction by exposure of the reaction mixture to light. The conditions of each reaction, such as temperature and pressure are effective, together with the reaction promoter used, if any, to obtain the selectivity to desired reaction product desired. Preferred selectivities are disclosed hereinafter for each of the reactions. Convenience may dictate that the reaction be carried out at ambient temperature (15° C. to 25° C.), and/or ambient pressure (0.7 to 1 Bar) to obtain the selectivity desired.

### Steps (a) and (b)

### Production of HCFC-336mdd

With respect to reacting step (a) and recovery step (b), both as part of the integrated process of the present invention and as a subcombination invention, the reaction of E-1,1,1,4,4,4-hexafluoro-2-butene (E-CF₃CH═CHCF₃) with chlorine to form dichloro-1,1,1,4,4,4-hexafluorobutane (CF₃CHClCHClCF₃) is a dichlorination reaction in which two moles of chlorine/per mol of E-1,1,1,4,4,4-hexafluoro-2-butene are reacted to obtain the desired HCFC-336mdd (CF₃CHClCHClCF₃) reaction product. The reaction can be carried out in a liquid medium or in the vapor phase, each preferably in the presence of a reaction promoter such as catalyst or photoinitiation. An example of liquid medium is the E-1,1,1,4,4,4-hexafluoro-2-butene (E-isomer) reactant itself. Examples of suitable reaction promoters include catalysts that cause the reaction to proceed ionically and photoinitiation that causes the reaction to proceed free radically. Examples of ionic directing catalysts include Lewis acids, such as transition metal chlorides or aluminum chloride. Photoinitiation causes homolysis of the chlorine reactant. Catalysis or photoinitation can be used in the liquid medium or vapor phase reaction.

The temperature and pressure conditions for the reaction are preferably selected to be effective to produce the HCFC-336mdd at high selectivity. In carrying out the reaction in the liquid phase such as supplied by the E-isomer itself, the reaction is preferably carried out in a closed pressurizable reactor within which the pressure is sufficient pressure to maintain the E-isomer or the HCFC-336mdd reaction product in the liquid state. The pressure within the reactor can be or include autogenous pressure. The desired reaction product HCFC-336mdd can be recovered from the reaction system when the reaction is carried out in a liquid medium by purging unreacted chlorine, distilling off unreacted E-isomer, and filtering off the catalyst.

A tubular reactor can be used to carry out the reaction in the vapor state (phase). Catalyst, such as Lewis acid, can be positioned within the reactor for effective contact with the E-isomer and chlorine gaseous reactants simultaneously fed into the reactor at a temperature and residence time effective to obtain the desired HCFC-336mdd reaction product in the selectivity desired. The temperature of the reaction is maintained by applying heat to the reactor. Preferably the temperature of the reaction is in the range of 100° C. to 200° C. The pressure within the tubular reactor is preferably about 0.1 to 1 MPa. The HCFC-336mdd reaction product can be recovered by distillation.

The conversion of the E-isomer to reaction product is preferably provides a selectivity to the formation of HCFC-336mdd of at least 85%, more preferably at least 90%, and most preferably, at least 95%, whether the reaction is carried out in the liquid phase or vapor phase.

### Steps (c) and (d)

### Production of 1,1,1,4,4,4-hexafluoro-2-butyne

With respect to reacting step (c) and recovery step (d), the reaction converting HCFC-336mdd to hexafluoro-2-butyne, wherein the HCFC-336mdd is twice dehydrochlorinated, is preferably carried out in a basic aqueous medium preferably in the presence of of a reaction promoter that is a catalyst. Preferably the basic aqueous medium comprise a solution of an alkali metal hydroxide or alkali metal halide salt or other base in water. Preferably the catalyst is a phase transfer catalyst. As used herein, phase transfer catalyst is intended to mean a substance that facilitates the transfer of ionic compounds into an organic phase, such as the HCFC-336mdd reactant, from an aqueous phase. The phase transfer catalyst facilitates the reaction of these dissimilar and incompatible components. While various phase transfer catalysts may function in different ways, their mechanism of action is not determinative of their utility in the present invention provided that the phase transfer catalyst facilitates the dehydrochlorination reaction.

A preferred phase transfer catalyst is quaternary alkylammonium salt. In one embodiment, at least one alkyl group of the quaternary alkylammonium salt contains at least 8 carbons. An example of quaternary alkylammonium salt wherein three alkyl groups contain at least 8 carbon atoms includes trioctylmethylammonium chloride (Aliquat® 336). An example of quaternary alkylammonium salt wherein four alkyl groups contain at least 8 carbon atoms includes tetraoctylammonium salt. The anions of such salts can be halides such as chloride or bromide, hydrogen sulfate, or any other commonly used anion. Specific quaternary alkylammonium salts include tetraoctylammonium chloride, tetraoctylammonium hydrogen sulfate, tetraoctylammonium bromide, methytrioctylammonium chloride, methyltrioctylammonium bromide, tetradecylammonium chloride, tetradecylammonium bromide, and tetradodecylammonium chloride. According to this embodiment, the phase transfer catalyst and reaction conditions are effective to achieve conversion of HCFC-336mdd preferably at least 50% per hour.

In another embodiment, the alkyl groups of the quaternary alkylammonium salt contain from 4 to 10 carbon atoms and a non-ionic surfactant is present in the aqueous basic medium. According to this embodiment, the phase transfer catalyst and reaction conditions are effective to achieve conversion of HCFC-336mdd preferably at least 20% per hour. The anions of quaternary alkylammonium salt wherein the alkyl group's salts contain 4 to 10 carbon atoms can be halides such as chloride or bromide, hydrogen sulfate, or any other commonly used anion. Quaternary alkylammonium salts mentioned above can be used in this embodiment provided their alkyl groups contain 4 to 10 carbon atoms. Specific additional salts include tetrabutylammonium chloride, tetrabutylammonium bromide, and tetrabutylammonium hydrogen sulfate.

Preferred non-ionic surfactants include ethoxylated nonylphenol or an ethoxylated C12-C15 linear aliphatic alcohol. Non-ionic surfactants include Bio-soft® N25-9 and Makon® 10 useful in the present invention are obtainable from Stepan Company.

In one embodiment, the quaternary alkylammonium salt is added in an amount of from 0.5 mole percent to 2.0 mole percent of the HCFC-336mdd. In another embodiment, the quaternary alkylammonium salt is added in an amount of from 1 mole percent to 2 mole percent of the HCFC-336mdd. In yet another embodiment, the quaternary alkylammonium salts is added in an amount of from 1 mole percent to 1.5 mole percent of the HCFC-336mdd. In one embodiment, the quaternary alkylammonium salt is added in an amount of from 1 mole percent to 1.5 mole percent of the HCFC-336mdd and the weight of non-ionic surfactant added is from 1.0 to 2.0 times the weight of the quaternary alkylammonium salt. These amounts apply to each of the above-mentioned embodiments of the quaternary alkylammonium salt used.

In each embodiment, the reaction is preferably conducted at a temperature of from about 60 to 90° C., most preferably at 70° C.

The basic aqueous medium is a liquid (whether a solution, dispersion, emulsion, or suspension and the like) that is primarily an aqueous liquid having a pH of over 7. In some embodiments the basic aqueous solution has a pH of over 8. In some embodiments, the basic aqueous solution has a pH of over 10. In some embodiments, the basic aqueous solution has a pH of 10-13. In some embodiments, the basic aqueous solution contains small amounts of organic liquids which may be miscible or immiscible with water. In some embodiments, the liquid medium in the basic aqueous solution is at least 90% water. In one embodiment the water is tap water; in other embodiments the water is deionized or distilled.

The base in the aqueous basic solution is selected from the group consisting of hydroxide, oxide, carbonate, or phosphate salts of alkali, alkaline earth metals and mixtures thereof. In one embodiment, bases which may be used lithium hydroxide, sodium hydroxide, potassium hydroxide, calcium hydroxide, magnesium oxide, calcium oxide, sodium carbonate, potassium carbonate, sodium phosphate, potassium phosphate, or mixtures thereof.

In one embodiment, the dehydrochlorination of dichloro-1,1,1,4,4,4-hexafluorobutane is conducted in the presence of an alkali metal halide salt. The alkali metal can be sodium or potassium. The halide can be chloride or bromide. A preferred alkali metal halide salt is sodium chloride. Without wishing to be bound by any particular theory, it is believed that the alkali metal halide salt stabilizes the phase transfer catalyst. Although the dehydrochlorination reaction itself produces alkali metal chloride, and in particular sodium chloride if sodium hydroxide is used as the base, addition of extra sodium chloride provides a further effect of increasing the yield of 1,1,1,4,4,4-hexafluoro-2-butyne. In one embodiment, the alkali metal halide is added at from 25 to 100 equivalents per mole of phase transfer catalyst. In another embodiment, the alkali metal halide is added at from 30 to 75 equivalents per mole of phase transfer catalyst. In yet another embodiment, the alkali metal halide is added at from 40 to 60 equivalents per mole of phase transfer catalyst. These amounts apply to each of the quaternary alkylammonium salts mentioned above.

As used herein, the basic aqueous solution is a liquid (whether a solution, dispersion, emulsion, or suspension and the like) that is primarily an aqueous liquid having a pH of over 7. In some embodiments the basic aqueous solution has a pH of over 8. In some embodiments, the basic aqueous solution has a pH of over 10. In some embodiments, the basic aqueous solution has a pH of 10-13. In some embodiments, the basic aqueous solution contains small amounts of organic liquids which may be miscible or immiscible with water. In some embodiments, the liquid medium in the basic aqueous solution is at least 90% water. In one embodiment the water is tap water; in other embodiments the water is deionized or distilled.

These embodiments of aqueous basic medium and bases apply to all of the phase transition catalysts, amounts, and reaction conditions mentioned above. The selectivity to the formation of 1,1,1,4,4,4,-hexafluoro-2-butyne is preferably at least 85%.

Additional details of the reacting step (c) are disclosed in PCT/US13/62080, filed Sep. 27, 2013 (docket designation FL1653), and the disclosure of this application is incorporated by reference herein.

This 1,1,1,4,4,4,-hexafluoro-2-butyne (boiling point −25° C.) can be recovered from the basic aqueous medium by distillation, wherein the butyne vaporizes from the aqueous medium and can then be condensed.

### Step (e)

### Production of Z-1,1,1,4,4,4-hexafluro-2-butene, Including Recovery

With respect to the reacting step (e) and recovery of the Z-1,1,1,4,4,4-hexafluoro-2-butene from this reaction, the reaction of hexafluoro-2-butyne with hydrogen to form said Z-1,1,1,4,4,4-hexafluoro-2-butene is preferably carried out in the presence of reaction promoter that is an alkyne-to-alkene catalyst.

One embodiment of alkyne-to-alkene catalyst is the palladium catalyst dispersed on aluminum oxide or titanium silicate, doped with silver and/or a lanthanide, with a low loading of palladium. In one embodiment, the palladium loading is from 100 ppm to 5000 ppm. In another embodiment, the palladium loading is from 200 ppm to 5000 ppm. In one embodiment, the catalyst is doped with at least one of silver, cerium or lanthanum. In one embodiment, the mole ratio of cerium or lanthanum to palladium is from 2:1 to 3:1. In one embodiment the mole ratio of silver to palladium is about 0.5:1.0.

Another embodiment of alkyne-to-alkene catalyst is the Lindlar catalyst, which is a heterogeneous palladium catalyst on a calcium carbonate support, which has been deactivated or conditioned with a lead compound. The lead compound can be lead acetate, lead oxide, or any other suitable lead compound. In one embodiment, the catalyst is prepared by reduction of a palladium salt in the presence of a slurry of calcium carbonate, followed by the addition of the lead compound. In one embodiment, the palladium salt in palladium chloride. In another embodiment, the catalyst is deactivated or conditioned with quinoline. The amount of palladium on the support is typically 5% by weight but may be any catalytically effective amount. In another embodiment, the amount of palladium on the support in the Lindlar catalyst is greater than 5% by weight. In yet another embodiment, the amount of palladium on the support can be from about 5% by weight to about 1% by weight.

In one embodiment, the amount of the catalyst used is from about 0.5% by weight to about 4% by weight of the amount of the 1,1,1,4,4,4-hexafluoro-2-alkyne. In another embodiment, the amount of the catalyst used is from about 1% by weight to about 3% by weight of the amount of the alkyne. In yet another embodiment, the amount of the catalyst used is from about 1% to about 2% by weight of the amount of the alkyne.

In some embodiments, the reaction of step (e) is conducted in a solvent. In one such embodiment, the solvent is an alcohol. Typical alcohol solvents include ethanol, i-propanol and n-propanol. In another embodiment, the solvent is a fluorocarbon or hydrofluorocarbon. Typical fluorocarbons or hydrofluorocarbons include 1,1,1,2,2,3,4,5,5,5-decafluoropentane and 1,1,2,2,3,3,4-heptafluorocyclopentane.

In one embodiment, the reaction is conducted in a batchwise process.

In another embodiment, the reaction is conducted in a continuous process in the gas phase.

In one embodiment, reaction of the 1,1,1,4,4,4-hexafluoro-2-butyne with hydrogen in the presence of the catalyst is preferably done with addition of hydrogen in portions, with increases in the pressure of the vessel of no more than about 100 psi (0.69 MPa)with each addition. In another embodiment, the addition of hydrogen is controlled so that the pressure in the vessel increases no more than about 50 psi (0.35 MPa) with each addition. In one embodiment, after enough hydrogen has been consumed in the hydrogenation reaction to convert at least 50% of the butyne to the desired butene (Z-1,1,1,4,4,4,-hexafluoro-2-butene), hydrogen can be added in larger increments for the remainder of the reaction. In another embodiment, after enough hydrogen has been consumed in the hydrogenation reaction to convert at least 60% of the butyne to the desired butene, hydrogen can be added in larger increments for the remainder of the reaction. In yet another embodiment, after enough hydrogen has been consumed in the hydrogenation reaction to convert at least 70% of the butyne to desired butene, hydrogen can be added in larger increments for the remainder of the reaction. In one embodiment, the larger increments of hydrogen addition can be 300 psi (2.07 MPa). In another embodiment, the larger increments of hydrogen addition can be 400 psi (2.76 MPa).

In one embodiment, the amount of hydrogen added is about one molar equivalent per mole of the butyne, 1,1,1,4,4,4-hexafluoro-2-butyne. In another embodiment, the amount of hydrogen added is from about 0.9 moles to about 1.3 moles, per mole of the butyne. In yet another embodiment, the amount of hydrogen added is from about 0.95 moles to about 1.1 moles, per mole of the butyne. In yet another embodiment, the amount of hydrogen added is from about 0.95 moles to about 1.03 moles, per mole of the butyne.

In one embodiment, the hydrogenation is performed at ambient temperature (15° C. to 25° C.). In another embodiment, the hydrogenation is performed at above ambient temperature. In yet another embodiment, the hydrogenation is performed at below ambient temperature. In yet another embodiment, the hydrogenation is performed at a temperature of below about 0° C.

In an embodiment of a continuous process, a mixture of 1,1,1,4,4,4-hexafluoro-2-butyne and hydrogen are passed through a reaction zone containing the catalyst. A reaction vessel, e.g., a metal tube, can be used, packed with the catalyst to form the reaction zone. In one embodiment, the molar ratio of hydrogen to the butyne is about 1:1. In another embodiment of a continuous process, the molar ratio of hydrogen to the butyne is less than 1:1. In yet another embodiment, the molar ratio of hydrogen to the butyne is about 0.67:1.0.

In one embodiment of a continuous process, the reaction zone is maintained at ambient temperature. In another embodiment of a continuous process, the reaction zone is maintained at a temperature of 30° C. In yet another embodiment of a continuous process, the reaction zone is maintained at a temperature of about 40° C.

In one embodiment of a continuous process, the flow rate of 1,1,1,4,4,4-hexafuro-2-butyne and hydrogen is maintained so as to provide a residence time in the reaction zone of about 30 seconds. In another embodiment of a continuous process, the flow rate of the butyne and hydrogen is maintained so as to provide a residence time in the reaction zone of about 15 seconds. In yet another embodiment of a continuous process, the flow rate of butyne and hydrogen is maintained so as to provide a residence time in the reaction zone of about 7 seconds.

It will be understood, that contact time in the reaction zone is reduced by increasing the flow rate of the butyne and hydrogen into the reaction zone. As the flow rate is increased this will increase the amount of butyne being hydrogenated per unit time. Since the hydrogenation is exothermic, depending on the length and diameter of the reaction zone, and its ability to dissipate heat, at higher flow rates it may be desirable to provide a source of external cooling to the reaction zone to maintain a desired temperature.

The conditions of the reacting step, including the choice of catalyst, are preferably selected to obtain Z-1,1,1,4,4,4-hexafluoro-2-butene at a selectivity of at least 85%, more preferably at least 90%, and most preferably at least 95%.

Each of the forgoing embodiments can be used in any combination in. the conduct of reacting step (e)

Additional details of the reacting step (e) are disclosed in U.S. Pat. No. 8,618,339 and the disclosure of this patent is incorporated by reference herein.

In one embodiment, upon completion of a batch-wise or continuous hydrogenation process, the Z-1,1,1,4,4,4-hexafluoro-2-butene can be recovered through any conventional process, including for example, fractional distillation. In another embodiment, upon completion of a batchwise or continuous hydrogenation process, the Z-1,1,1,4,4,4-hexafluoro-2-butene is of sufficient purity to not require further purification steps.

### Back Integration (A)

### Production, Including Recovery, of E-1,1,1,4,4,4-hexafloro-2-butene, The Starting Material for Step (a)

With respect to back integration of the process to include the reaction to obtain E-1,1,1,4,4,4-hexafluoro-2-butene (E-isomer) as the starting material for the integrated process to convert this starting material to the Z-isomer, the process to obtain the E-isomer preferably comprises reacting HCFC-123 with copper in the presence of an amide solvent and catalyst, which can be (i) 2,2′-bipyridine, (ii) Cu(I) salt, or (iii) both (i) and (ii).

HCFC-123 is commercially available from E. I. du Pont de Nemours and Company incorporated in Delaware.

Copper used herein is metal copper having zero valence. In one embodiment of this invention, copper powder is used for the reaction.

Typical amide solvents used herein include dimethylformamide (DMF), dimethylacetamide, N-methylpyrrolidone, et al. In one embodiment of this invention, the amide solvent is DMF.

Typical Cu(I) salts used herein include CuCI, CuBr, CuI, copper(I) acetate, et al. In one embodiment of this invention, the Cu(I) salt is CuCI.

Optionally, an amine can also be present in the reaction mixture. Typically such amines include secondary amines such as dimethylamine, diethylamine, di-n-propylamine, diisopropylamine, di-n-butylamine, et al.; tertiary amines such as trimethylamine, triethylamine, tri-n-propylamine, triisopropylamine, tri-n-butylamine, et al.; cyclic amines such as morpholine, piperazine, piperidine, pyrrolidine, et al.

Both the 2′2-bipyridene and Cu(I) salt contribute to the selectivity of the converted HCFC-123 to form E/Z-1,1,1,4,4,4-hexafluoro-2-butene. Selectivity of these isomers as a mixture is preferably at least 90%, more preferably at least 95%. The amount of each isomer formed is about 50% of the isomer mixture.

The reaction can be carried out at ambient temperature and in a closed vessel to capture the vaporized reaction products.

Additional details of this reaction is disclosed in U.S. Pat. No. 8,436,216, the disclosure of which is incorporated by reference herein.

The E and Z isomers can be recovered from the reaction product and from each other by fractional distillation. The recovered Z-isomer can be added to the Z-isomer recovered from step (e) of the integrated process.

### Back Integration (B)

### Production, Including Recovery, of E-1,1,1,4,4,4-hexafloro-2-butene, The Starting Material for Step (a)

In this back integration, the E-1,1,1,4,4,4-hexafluoro-2-butene starting material for step (a) is obtained by (i) reacting 3,3,3-trifluoroprop-1-ene with carbon tetrachloride to form 2,4,4,4-tetrachloro-1,1,1-trifluorobutane and (ii) fluorinating said 2,4,4,4-tetrachloro-1,1,1-trifluorobutane to form said E-1,1,1,4,4,4-hexafluoro-2-butene.

With respect to reaction (i), this reaction is preferably carried out in the presence of a catalyst at an elevated temperature, whereby the reaction is carried out in the gas phase. A preferred catalyst is the combination of iron powder with tributyl phosphate, and the temperature is chosen to drive the reaction to completion. Preferably, the temperature is in the range of 75° C. to 150° C. For convenience, the reaction can be run under autogenous pressure. The 2,4,4,4-tetrachloro-1,1,1-trifluorobutane reaction product can be recovered from the reaction product mixture by distillation, thereby making the recovered 2,4,4,4-tetrachloro-1,1,1-trifluorobutane available to be the starting material for reaction (ii).

With respect to reaction (ii), this reaction is preferably carried out using HF as the fluorinating agent, i.e. the 2,4,4,4-tetrachloro-1,1,1-trifluorobutane is reacted with the HF. The HF is preferably used as a mixture with nitrogen. This reaction is preferably carried out in the vapor phase and in the presence of a catalyst. A preferred catalyst is activated chromium oxide on carbon on. This reaction is also preferably carried out at a temperature in the range of 250° C. to 350° C. The reaction can be carried out under autogenous pressure or a pressure in the range of 0 to 3.4 MPa. The E-1,1,1,4,4,4-hexafluoro-2-butene can be recovered from the reaction product mixture by distillation to make the E-1,1,1,4,4,4-hexafluoro-2-butene available as the starting material for step (a).

The various embodiments of each reaction and recovery described above can be used in any combination in the integrated process of the present invention.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. In case of conflict, the present specification, including definitions, will control. In addition, the materials, methods, and examples are illustrative only and not intended to be limiting.

### EXAMPLES

The concepts described herein will be further described in the following examples, which do not limit the scope of the invention described in the claims.

### Example 1

### E-1,1,1,4,4,4-hexafluoro-2-butene (E-1336mzz, E-isomer)) to CF₃CHClCHClCF₃ (HCFC-336mdd)

In this Example, the E-isomer is catalytically thermally chlorinated either in the liquid phase or the vapor phase to form HCFC-336mdd. Lewis acid catalysts are used. Ferric Chloride, chromium chloride, alumina chloride, cupric chloride catalysts and chlorine are available from Sigma Aldrich, St. Louis, Mo. E-1336mzz is available from Synquest Labs, Inc.

The liquid phase reaction was carried out in a Hast® C reactor. The liquid was the E-isomer reactant. Catalyst when used was present in the liquid phase. The reactor content was transferred to a cylinder and analyzed by GC to determine the conversion and selectivity. The HCFC-336mdd was recovered from the reaction by purging unreacted chlorine, distilling off the unreacted E-isomer and filtering off the catalyst. Reaction conditions and results are given in Table 1.

TABLE 1
Liquid Phase Thermal Chlorination of E-1336mzz

| | | | | Conversion/ |
|---|---|---|---|---|
| Examples | Catalyst | T (° C.) | t (hr) | Selectivity (%) |
| 1-1 | FeCl3 | 150 | 0.5 | 60/100 |
| 1-2 | CrCl3 | 150 | 1 | 60/87.3 |
| 1-3 | AlCl3 | 150 | 2 | 69/97.6 |
| 1-4 | CuCl2 | 150 | 2 | 60/98 |
| 1-5 | None | 180 | 2 | 63/40 |

For each of Examples 1-1 to 1-4, E-1336mzz (20 g, 0.122 mole) and chlorine (8.65 g, 0.122 mole) were heated to 150° C. in the presence of FeCl₃, CrCl₃, AlCl₃ or CuCl₂ catalyst (0.4 g, 0.0025 mol) in the Hast® C reactor for 2 hrs.

For Example 1-5, the E-1336mzz (20 g, 0.122 mole) and chlorine (8.65 g, 0.122 mole) were heated to 180° C. in a 210 mL Hast® C reactor for 2 hrs. No catalyst was present.

Comparison of the results for Examples 1-1 to 1-4 with 1-5 indicates the preference for the reaction being carried out in the presence of catalyst.

The procedure for the gas phase reaction was as follows: An Inconel tube® (0.5 inch OD, 15 inch length, 0.34 in wall thickness) was filled with 2 cc (1.10 gm) of ferric chloride on acid washed Takeda® carbon. The reactor was heated in a Lindberg furnace to 125° C. and CF₃CH═CHCF₃ (E-1336mzz) was fed at 2.42-4.83 ml/hour and chlorine gas at 6.2-13.0 sccm (standard cubic centimeters per minute) through a vaporizer controlled at 80° C. Over the course of the run, the temperature was raised to 175° C. All of the experiments below were carried out at 49-51 psig (0.34 -0.35 MPa). The effluent of the reactor was analyzed online using an Agilent® 6890 GC/5973 MS and a Restek® PC2618 5% Krytox® CBK-D/60/80 6 meter×2 mm ID ⅛″ OD packed column purged with helium at 30 sccm. The HCFC-336mdd was recovered by distillation.

The data is shown in the tables, and samples are taken in hourly intervals.

TABLE 2
Gas Phase Chlorination of E-1336mzz

| Mole Percents | | | | | | | | | | | | |
|---|---|---|---|---|---|---|---|---|---|---|---|---|
| Unknowns | 236fa | 1336 | 133a | 123 | 336 | Furnace | Pressure | Pump | Cl2 | C T | Conv | Sel |
| % | % | % | % | % | % | C. | (MPa) | ml/hr | sccm | sec | % | % |
| 0.69 | 0.00 | 92.91 | 4.13 | 0.33 | 1.94% | 125 | 0.349 | 4.83 | 12.96 | 14 | 2.8 | 73.9 |
| 1.06 | 0.12 | 92.70 | 4.02 | 0.37 | 1.73% | 125 | 0.349 | 4.83 | 12.96 | 14 | 2.9 | 62.1 |
| 1.11 | 0.12 | 92.74 | 4.07 | 0.36 | 1.62% | 125 | 0.342 | 4.83 | 12.96 | 14 | 2.9 | 59.4 |
| 1.15 | 0.00 | 92.90 | 4.08 | 0.35 | 1.52% | 125 | 0.342 | 4.83 | 12.98 | 14 | 2.8 | 56.9 |
| 0.97 | 0.00 | 90.46 | 4.11 | 0.36 | 4.10% | 150 | 0.349 | 4.83 | 12.96 | 14 | 5.3 | 80.9 |
| 0.94 | 0.12 | 90.47 | 4.10 | 0.36 | 4.01% | 150 | 0.349 | 4.83 | 12.95 | 14 | 5.2 | 81.0 |
| 0.86 | 0.12 | 90.58 | 4.06 | 0.37 | 4.01% | 150 | 0.356 | 4.83 | 12.97 | 14 | 5.1 | 82.3 |
| 0.61 | 0.00 | 84.53 | 4.04 | 0.38 | 10.44% | 175 | 0.377 | 4.83 | 10.67 | 17 | 11.6 | 94.5 |
| 0.58 | 0.00 | 85.82 | 3.99 | 0.39 | 9.22% | 175 | 0.333 | 4.83 | 12.98 | 13 | 10.3 | 94.1 |
| 0.70 | 0.00 | 84.74 | 3.91 | 0.38 | 10.27% | 175 | 0.337 | 4.83 | 12.96 | 14 | 11.5 | 93.7 |
| 0.58 | 0.12 | 84.54 | 4.08 | 0.37 | 10.33% | 175 | 0.337 | 4.83 | 12.96 | 14 | 11.4 | 94.7 |
| 0.58 | 0.12 | 84.65 | 4.02 | 0.37 | 10.26% | 175 | 0.337 | 4.83 | 12.96 | 14 | 11.4 | 94.7 |
| 0.10 | 0.13 | 82.97 | 4.75 | 0.37 | 11.67% | 175 | 0.344 | 2.42 | 6.26 | 28 | 12.4 | 99.1 |
| 0.10 | 0.11 | 80.94 | 4.43 | 0.39 | 14.02% | 175 | 0.357 | 2.42 | 6.24 | 29 | 14.9 | 99.3 |
| 0.11 | 0.13 | 77.91 | 4.89 | 0.40 | 16.57% | 175 | 0.355 | 2.42 | 6.24 | 29 | 17.6 | 99.4 |
| 1.08 | 0.00 | 88.74 | 3.91 | 0.37 | 5.91% | 150 | 0.344 | 2.42 | 6.25 | 28 | 7.3 | 84.6 |
| 0.80 | 0.00 | 89.64 | 4.02 | 0.36 | 5.27% | 150 | 0.323 | 2.42 | 6.24 | 27 | 6.3 | 86.5 |
| 0.77 | 0.00 | 88.51 | 3.76 | 0.40 | 4.56% | 150 | 0.377 | 2.42 | 6.25 | 28 | 7.6 | 89.5 |
| 0.90 | 0.12 | 91.61 | 4.04 | 0.34 | 3.10% | 125 | 0.344 | 2.42 | 6.25 | 28 | 4.1 | 77.0 |
| 0.98 | 0.11 | 92.29 | 4.06 | 0.36 | 2.20% | 125 | 0.344 | 2.42 | 6.24 | 28 | 3.3 | 69.2 |
| 0.99 | 0.12 | 92.38 | 4.05 | 0.37 | 2.19% | 124 | 0.344 | 2.42 | 6.25 | 28 | 3.2 | 67.8 |

In Table 2, 236fa and 123 are impurities in the feed to the reactor.

The reaction conditions producing 27 to 29 sec. contact time at reactor temperature of 175° C. produces the best selectivities in the production of HCFC-336mdd.

### Example 2

### E-1,1,1,4,4,4-hexafluoro-2-butene to CF₃CHClCHClCF₃ (HCFC-336mdd)

In this Example, the reaction is photoinitiated.

A 50 gallon (190 L) stirred reaction vessel equipped with a column, overhead condenser, dip-tube, and quartz light-well with a cooling jacket. The light-well fitted with a 450 watt mercury arc-lamp bulb.

To this reactor was charged 158 Kg of E1336mzz and this liquid was cooled to 0° C. The agitator on running a 100 rpm and the overhead condenser cooled to ˜−20° C. the light was turned on. To this system 69 Kg of chlorine was slowly added through the dip-tube over 51 hours using the feed rate to control temperature and pressure. The liquid reaction temperature and pressure were not allowed to go above 10° C. and 1 psig (0.07 MPa), respectively.

On completion of the chlorine addition, the light was turned off and the solution was allowed to warm to room temperature. The system was vented to ambient through a caustic scrubber and the crude reaction mixture was de-inventoried to a storage vessel. Recovery of the HCFC-336mdd was carried out by combining 3 batches of the resulting crude reaction mixture (663 Kg/422 L) and then added slowly adding the crude reaction mixture through a dip-tube to a 200 gallon (750 L)stirred vessel equipped with bottom discharge valve and charges with 80 gallons (300 L) of an aqueous solution of 10% K₂HPO₄/KH₂PO₄. After the addition was done this mixture was vigorously stirred for 3 hours and the agitation was then turned off. The lower organic phase was then decanted from the reactor using conductivity measurements to determine the change in phase. The resulting neutralized organic oil was a water-white liquid and had a pH of 5-6 was passed through a bed of molecular sieves to dry it and stored for final purification. Isolated chemical yield over 7 batches was 98%. The resulting GC assay (% FID) was 93.5% of the two 336mdd diastereomers with the balance of the assay being heavy unknowns ˜6% presumed to be oligomers of the product/starting materials, whereby the selectivity of the reaction was 93.5% Final purification was done by distillation.

### Example 3

### Integrated Process

The E-1,1,1,4,4,4-hexafluoro-2-butene (E-isomer) was obtained by conversion from HCFC-123 to obtain a mixture of the E-isomer with the Z-isomer (Z-1,1,1,4,4,4-hexafluoro-2-butune) by the following procedure:

At room temperature, a 80 ml Fisher Porter tube was charged with 1.85 g (0.029 mol) of Cu powder, 2 g (0.013 mol) of HCFC-123 (b. pt 28° C.), 0.15 g (0.0015 mol) of CuCl, 0.3 g (0.0019 mol) of 2,2′-bipyridine and 10 ml of DMF. The tube was purged with N₂ for 5 minutes and then was sealed. The reaction mixture was stirred at 80° C. for 4 hours. The pressure of the tube increased to 10.5 psig (0.072 MPa) at 80° C. It dropped to 4.5 psig (0.03 MPa) after the tube was cooled down to room temperature. At the end of the reaction, both vapor phase and liquid phase of the product mixture in the tube were analyzed by GC-MS. The analytical results were given in units of GC area % in Table 1 and Table 2 below. Small amounts of byproducts having GC area % less than 0.05 were not included in the Tables.

TABLE 3
(vapor phase)

| E-isomer | Z-isomer | CF₃CH═CHCF₂Cl | HCFC-123 |
|---|---|---|---|
| 82.62 | 13.93 | 0.18 | 3.23 |

TABLE 4
(liquid phase)

| E-isomer | Z-isomer | CF₃CH═CHCF₂Cl | HCFC-123 | unknowns |
|---|---|---|---|---|
| 46.88 | 49.71 | 0.65 | 2.40 | 0.33 |

The selectivity of the formation of the E/Z-isomer mixture from the HCFC-123 was in excess of 95%. The E-isomer first separated from the DMF and its contents by distillation. The Z-isomer was next separated from the DMF and its contents by distillation.

The E-isomer obtained from the conversion of HCFC-123 and recovery as described above was next reacted with chlorine to form HCFC-336mdd using the procedure for the gas phase process described under Example 1 in accordance with the specific information in Table 2 to provide the selectivity of HCFC-336mdd of 99.4%.

The HCFC-336mdd obtained by the procedure disclosed in the preceding paragraph was next used in the reaction with base to form 1,1,1,4,4,4,-hexafluoro-2-butyne using the following procedure: NaOH aqueous solution (22 mL, 0.22 mole) was added to the 336mdd (23.5 g, 0.1 mol) and water (5.6 mL) in the presence of Aliquat® 336 (0.53 g, 0.001325 mol), which is trioctylmethylammonium chloride, at room temperature ° C. The reaction temperature was raised to 70° C. after the addition, and gas chromatography was used to monitor the reaction. The reaction was completed after 2 hour and 14 g 1,1,1,4,4,4,-hexafluoro-2-butyne product (conversion: 100%; yield: 86%) was collected in a dry ice trap. The butyne was purified by distillation.

The 1,1,1,4,4,4-hexafluoro-2-butyne obtained by the procedure disclosed in the preceding paragraph was next reacted with hydrogen to obtain the desired Z-isomer of 1,1,1,4,4,4-hexafluoro-2-butene by the following procedure: 5 g of Lindlar (5 % Pd on CaCO₃ poisoned with lead) catalyst was charged in 1.3 L rocker bomb. 480 g (2.96 mole) of hexafluoro-2-butyne was charged in the rocker. The reactor was cooled down (−78° C.) and evacuated. After the bomb was warmed up to room temperature, H₂ was added slowly, by increments which did not exceed Δp=50 psi (0.35 MPa). A total of 3 moles H₂ were added to the reactor. A gas chromatographic analysis of the crude product indicated the mixture consisted of CF₃C≡CCF₃ (0.236%), trans-isomer E-CF₃CH═CHCF₃ (0.444%), saturated CF₃CH₂CH₂CF₃ (1.9%) CF₂═CHCl, impurity from starting butyne, (0.628%), cis-isomer Z—CF₃CH═CHCF₃ (96.748%). Distillation afforded 287 g (59% yield) of 100% pure cis-CF₃CH═CHCF₃ (boiling point 33.3° C.). MS: 164 [MI], 145 [M-19], 95 [CF₃CH═CH], 69 [CF₃]. NMR H¹: 6.12 ppm (multiplet), F¹⁹: −60.9 ppm (triplet J=0.86 Hz). The selectivity of this reaction to the formation of the Z-isomer was 96.98%. The Z-isomer was recovered by distillation.

### Example 3

### Integrated Process Starting with the E-Isomer from a Different Source

In a 600 mL autoclave that is equipped with an agitator, thermocouple, relief valves, sample valves and a pressure gauge, about 200 mL carbon tetrachloride, 6 gram (0.11 mole) of iron powder and 6 grams (0.023 mole) of tributyl phosphate (TBP) are added to form a mixture. The reactor is cooled down to −50° C. by placing the autoclave in a dry ice/acetone bath, and a vacuum is pulled. To the mixture, 3,3,3-trifluoroprop-1-ene is added to develop an internal pressure of 3.4 kPa for the reaction mixture. The reaction mixture is heated to 105° C. thereby producing an autogenous pressure of about 7.7 kPa. The reaction mixture is maintained at temperature for several hours during which time the reactor pressure is observed to decrease and settle at 2.4 kPa. To the reaction mixture, additional 3,3,3-trifluoroprop-1-ene is fed discretely several times to drive the reaction to completion. The reaction is observed to be complete when the absence of a pressure decrease is evident. The contents of the autoclave is transferred and distilled to afford the 2,4,4,4-tetrachloro-1,1,1-trifluorobutane product having a boiling point of 86° C. at 163 mmHg. The product structure is confirmed by NMR and GCMS analysis.

The 2,4,4,4-tetrachloro-1,1,1-trifluoro-butane is next converted to the E-isomer according t the following procedure: The reactor has an outside diameter of 19 mm and a length of 600 mm and is composed of an Inconel® alloy. The reactor is equipped with a heater, thermocouple, product trap, and a syringe pump, and a catalyst system of activated chromium oxide on carbon is added to the reactor to form a reactor space. The reactor space is heated to from 350° C. and exposed to N₂ and a HF/N₂ mixture for 16 hours. The reactor space is cooled and maintained at 300° C. 270 mL/minute of gaseous HF and 11.4 mL/hour of liquid 2,4,4,4-tetrachloro-1,1,1-trifluorobutane is delivered into the reaction space through the syringe pump and into a pre-heater to vaporize the 2,4,4,4-tetrachloro-1,1,1-trifluorobutane to form a reaction mixture. The reaction mixture is washed with water, dried over MgSO₄ and collected in a dry-ice condenser to afford the crude E-1,1,1-4,4,4-hexafluoro-2-butene (E-isomer) product. Distillation of the crude product yielded 99% pure product having a boiling point of 8° C. The product structure is confirmed by NMR and/or GCMS analysis.

The E-isomer is next converted to HCFC-336mdd, which is then converted to 1,1,1,4,4,4-hexafluorbutyne, which is next converted to form 100% pure Z-1,1,1,4,4,4-hexafluoro-2-butene (Z-isomer) all in accordance with the reactions and reaction conditions disclosed in Example 3.

1-8. (canceled)

9. The process for obtaining dichloro-1,1,1,4,4,4-hexafluorobutane, comprising contacting E-1,1,1,4,4,4-hexafluorobutene with chlorine in either the gas phase, or the liquid phase.

10. The process of claim 9 wherein said contacting is carried out in the presence of catalyst selected from the group consisting of ferric chloride, chromium chloride, alumina chloride and cupric chloride.

11. The process of claim 10 wherein the selectivity for the production of dichloro-1,1,1,4,4,4-hexafluorobutane is at least 85%.

12. The process of claim 10, wherein said contacting is carried out at a temperature of from 125 C to 175 C.

13. The process of claim 9, wherein the process is conducted with exposure to ultraviolet light.

14. The process of claim 13 wherein the selectivity for the production of dichloro-1,1,1,4,4,4-hexafluorobutane is at least 85%.

15. The process of claim 13 wherein the selectivity for the production of dichloro-1,1,1,4,4,4-hexafluorobutane is at least 90%.
